(19) United States
(12) Patent Application Publication
(10) Pub. No.: US 2025/0161173 A1
(43) Pub. Date: May 22, 2025

(54) TABLETED EFFERVESCING DENTAL TREATMENT COMPOSITION, AND METHODS

(71) Applicant: Teeth Tabs PLLC, Goddard, KS (US)

(72) Inventors: Robert Harry DAKIN, Wichita, KS (US); Jeffrey Michael DAKIN, Goddard, KS (US)

(21) Appl. No.: 18/950,905

(22) Filed: Nov. 18, 2024

Related U.S. Application Data
(60) Provisional application No. 63/600,121, filed on Nov. 17, 2023.

(51) Int. Cl. A61K 8/04; A61K 8/02; A61K 8/19; A61K 8/365; A61K 8/73; A61K 8/9728; A61K 8/9789; A61K 8/9794; A61Q 11/00

(52) U.S. Cl. CPC A61K 8/046; A61K 8/0216; A61K 8/19; A61K 8/365; A61K 8/732; A61K 8/9728; A61K 8/9789; A61K 8/9794; A61Q 11/00

(57) ABSTRACT

A tableted effervescing dental treatment composition which provides for refreshing and/or dental plaque reduction would introduce, maintain within the oral cavity, which treatment composition is ingestible, and which forms a foam after introduction into the mouth. Methods for the manufacture of the effervescing dental treatment composition and treatment methods using the same are also disclosed.

The present invention relates to a tableted effervescing dental treatment composition, its method of manufacture and methods relating to the use of such compositions.

The maintenance of good oral health begins with a healthy oral cavity (buccal cavity). Such is effectively achieved by most persons in good health, by a regular regimen of brushing, flossing, and preferably also the use of an oral rinse products, i.e, a mouthwash. Such a regimen is useful in removal of plaque, and minimization of unwanted microbial growth on teeth, gums and other surfaces of the oral cavity, and thereby maintaining proper oral hygiene. Such is not only beneficial to overall health, but also is advantageous in interactions with others as a clean oral cavity is attractive, and less subject to halitosis or malodorous emissions, collectively “bad breath”. While most individuals are physically capable of undertaking such a regular regimen, there are individuals with compromised health, be it physical health, and or mental health, who are unable to practice such a beneficial regular regimen for maintaining proper oral hygiene. In some instances also, normally healthy individuals may be found in environments or circumstances where in their usual regular regimen of brushing, flossing, Thus there is real and present need for improved dental treatment compositions which may be used by such individuals.

In one aspect of the invention there is provided a solid, i.e., tableted effervescing dental treatment composition which when retained within the oral cavity, for a short period of time, will effervesce and deliver to surfaces present within the oral cavity a combination of active ingredients which comes into contact with tooth enamel and gingiva In further aspect the invention is provided method of treatment of teeth, and in particular tooth enamel, as well as gingiva, present in the oral cavity by the administration of a tableted effervescing dental treatment composition by oral delivery and oral retention for sufficient period of time to allow for effervescing and/or breakdown of the tableted dental treatment composition to thereby distribute active ingredients within the oral cavity. And yet further aspect, the invention provides a method of the manufacture of a tableted effervescing dental treatment composition which provides the foregoing treatment benefits. These further aspect of the invention will become more apparent from a reading of following specification.

A first aspect the present invention provides a tableted effervescing dental treatment composition which provides for refreshing and/or dental plaque reduction would introduce, maintain within the oral cavity, which treatment composition is ingestible, and which forms a foam after introduction into the mouth.

A second aspect of the present invention provides a method of providing a treatment benefit to oral surfaces, in particular the reduction of plaque upon dental surfaces within the mouth, the method comprising the step of providing a tableted effervescing dental treatment composition as hereinafter described, and maintaining it within the oral cavity for sufficient time in order to provide reduction in dental plaque and/or to provide a freshening benefit to the oral cavity.

A third aspect of the present invention is directed toward the method of producing a tableted effervescing dental treatment composition, useful in providing a treatment benefit within the oral cavity, and in particular to dental services within the mouth.

Further aspects of the invention will become apparent from a consideration of the following specification.

The tableted effervescing dental treatment compositions represent a preferred embodiment of the invention but, it is not required that the compositions be necessarily formed into compacted tablets. Rather, they may be provided in any form which allows for their placement within the oral cavity. For example, compositions described with reference to one or more tableted effervescing dental treatment composition may take the shape of pearls, pastilles, noodles, semi agglomerated or agglomerated powders, as well as free-flowing particulates in a non-agglomerated or semi agglomerated form. In its most beneficial format however, the tableted effervescing dental treatment compositions are three-dimensional bodies which comprise a quantity of the effervescent dental treatment composition, which bodies of such a size that they may be easily packaged either in a unitary package, or in a package containing a plurality of such three-dimensional bodies in a container which allows dispensing one or more of the same there from. In all embodiments, it is only necessary that the tableted effervescing dental treatment composition be in a form which can be delivered into the oral cavity. With this realization, the form of tablets (or pastilles) are particularly convenient in allowing for the production of a three-dimensional body, containing a quantity of the dental treatment composition, or alternately being composed entirely or substantially of the dental treatment composition, which body is conveniently placed into the mouth. Said three-dimensional bodies may take any shape or form, but generally forms which have a maximum dimension of about 2.5 cm, per preferably less are preferred.

The effervescing dental treatment compositions necessarily include a gas generator system which provides an effervescent effect, namely the evolution of a gas from a liquid resulting from a chemical reaction. This reaction can be between, for example, an acid and a metal carbonate, to produce carbon dioxide gas. Any gas generator system may be used in the compositions of the invention, as long as such are ingestible by humans and are not deleterious to human health.

Preferably the gas generator system comprises both an acid component which is an acid or acid source, which in the presence of water is capable of reacting with an alkali component which is an alkali or an alkali source in order to produce a gas. Preferably the resultant gas is oxygen, nitrogen dioxide or carbon dioxide but any other gas may also be formed.

The acid component may be any organic, mineral or inorganic acid, or mixtures thereof. Preferably the acid component is an ingestible organic acid. The acid component is preferably substantially anhydrous or non-hygroscopic and the acid is preferably also water-soluble or water dispersible. Exemplary useful acids include acetic acid, citric acid, tartaric acid, malic acid, succinic acid, folic acid, fumaric acid, and lactic acid. Preferred is citric acid, which is both effective and widely commercially available.

The alkali component may be any suitable alkali or alkali source which has the capacity to react with the acid component when contacted with water to produce a gas. The alkali component is also an ingestible material, which reacts with the acid component in order to release the gas in the presence of moisture. Exemplary alkali components include alkali metal carbonates, e.g, sodium carbonate, potassium carbonate, bicarbonate, sesquicarbonate, as well as perborates and percarbonates, as well as thereof. Preferred alkali components are edible bases, of which alkali metal carbonates, especially sodium bicarbonate are preferred for use.

The relative amounts of the acid components to the alkali component may vary widely, however preferably the respective molar ratios of the acid component to the alkali component be within the range of 1-5:1-5, preferably in respective molar ratios of 1-2.5:1-2.5, more preferably in the range of the former to the latter of 1-1.5:1-1.5. Most preferably the acid component to alkali component be present in approximately equal molar ratios to ensure the evolution of the gas. In certain further particularly preferred embodiments the acid component, especially where the acid component is citric acid, is present in a molar excess such at that the respective molar ratio of the acid component to the alkali component is at least 1-2:1. Alternately the delivery of an excess of the alkali component, such as at the respective molar ratio of the acid component to the alkali component of 1:1-2, or more, which excess may advantageously decrease the pH of surfaces within the oral cavity, as well as neutralizing the naturally acidic saliva present. Especially preferred as the alkali component is a bicarbonate salt, particularly preferably sodium bicarbonate.

The total amount of the alkali component comprised in the effervescing compositions is about 5-45% wt., preferably between 7-35% wt., with particularly preferred amounts disclosed in one or more the following examples.

The total amount of the acid component comprised in the effervescing compositions is 1-25% wt., preferably between 2.5-15% wt., with particularly preferred amounts disclosed in one or more the following examples.

A preferred gas generator system comprises two or more organic acids, in combination with one or more alkali components. By way of nonlimiting example a preferred gas generator system is a ternary systems which comprises citric acid:tartaric acid:sodium bicarbonate. Particularly preferred gas generator systems are disclosed with reference to one or more of the examples.

A next essential constituent of the tableted effervescing dental treatment compositions include an edible oil, or blend of edible oils. Such oils may function as one or more aspects including as a solution medium or a matrix for further constituents present. Nonlimiting examples of such useful oils include synthetic oils, semi-synthetic oils or natural oils including but not limited to soybean oil, sesame oil, peanut oil, olive oil, coconut oil, castor oil, sunflower oil, safflower oil, a mixture of at least two of the above oils or the corresponding hardened oil. Preferably the one or more oils are natural oils or blends thereof, especially comprising one or more of olive oil and coconut oil.

The total amount of the edible oil constituents comprised in the effervescing dental treatment compositions is advantageously of from about 22-33% wt., more preferably from about 25-30% wt., very preferably 27-28% wt., especially about 27.5% wt. Further particularly preferred amounts may be disclosed in one or more the following examples.

A further essential constituent is a natural starch constituent, preferably corn starch (cornstarch, maize starch), potato starch, rice starch, wheat starch, sweet potato starch, mung bean starch and tapioca starch. These natural starches may be used alone or in combination of two or more. Among these, corn starch, potato starch, wheat starch, and rice starch are preferred.

Corn starch is particularly preferred in providing good physical properties to tableted dosage forms of the effervescing dental treatment compositions.

The total amounts of the natural starch constituent comprised in the effervescing dental treatment compositions is advantageously of from about 8-19% wt., more preferably from about 11-16% wt., very preferably 13-14% wt., especially about 13.5% wt. Further particularly preferred amounts may be disclosed in one or more the following examples.

A further essential constituent is aloe vera (Aloe barbadensis), which may be used in either a liquid form or in a powdered form.

The total amount of the aloe vera comprised in the effervescing compositions from about 8-19% wt., more preferably from about 11-16% wt., very preferably 13-14% wt., especially about 13.5% wt. Further particularly preferred amounts may be disclosed in one or more the following examples. The aloe vera constituent is advantageously 90-100% wt. actives.

A further essential constituent is Shitake mushroom, advantageously in a dry powder form.

The total amount of the Shitake mushroom comprised in the effervescing compositions is from about 16-27% wt., more preferably from about 19-24% wt., very preferably 21-22% wt., especially about 21.1% wt. Further particularly preferred amounts may be disclosed in one or more the following examples.

A further essential constituent is cranberry extract, which is advantageously provided as a liquid, which may be a distillate or concentrate formed from cranberry fruits.

The total amount of the cranberry extract comprised in the effervescing compositions is from about 8-19% wt., more preferably from about 11-16% wt., very preferably 13-14% wt., especially about 13.5% wt. Further particularly preferred amounts may be disclosed in one or more the following examples.

A further essential constituent is black tea leaves, advantageously in a dry powder form.

The total amount of the black tea leaves comprised in the effervescing compositions is from about 0.01-8% wt., more preferably from about 0.1-5% wt., very preferably 2-3% wt., especially about 2.7% wt. Further particularly preferred amounts may be disclosed in one or more the following examples.

Optionally the tableted effervescing dental treatment compositions may include one or more further constituents which provide either technical and/or anesthetic benefit thereto, and may be any of a number of pharmaceutically acceptable excipients. Such include one or more of a flavorant, sweetener, colorant, binder, lubricant and disintegrant. Additionally one or more ancillary starches, other than the natural starches disclosed amongst the essential constituents may be present. However in accordance with certain preferred embodiments, one or more of these optional further constituents may be expressly excluded from the tableted effervescing dental treatment compositions.

Non-limiting examples of starches include starch and various chemically and mechanically processed starches (e.g., pregelatnized starch, maltodextrin) and hydroxypropylcellulose, and the like. Such may be present in addition to the essential, natural starch constituent. If present, the starch is most advantageously corn starch (maize starch), and in certain preferred embodiments the corn starch is the sole starch constituent present. When present, the optional starch provides 0.5-45% wt., preferably 1-25% wt. of the total of the effervescing dental treatment compositions. Preferred starches and preferred amounts of starches are disclosed with reference to one or more of the examples.

Non-limiting examples of sweeteners include sugar, honey, aspartame, saccharin, saccharin sodium, and dipotassium glycyrrhizinate, as well as high-intensity sweeteners such as stevia, thaumatin, sucralose, acesulfame K, and the like. Among them, preferred high-intensity sweeteners include aspartame, thaumatin and sucralose. When present, the optional sweetener provides 0.01-10% wt., preferably 0.05-8% wt. of the total of the effervescing dental treatment compositions.

Non-limiting examples of flavoring agents and flavoring agents include synthetic or naturally sourced materials, i.e., peppermint, spearmint, menthol, lemon, lemon, lime, grapefruit, strawberry, peach, blueberry, orange, grapefruit, pine, fruit and extracts thereof. When present, the optional flavorant provides When present, the optional starch provides 0.5-45% wt., preferably 1-25% wt. of the total of the effervescing dental treatment compositions. Preferred amounts of, and preferred optional flavorants are disclosed with reference to one or more of the examples. Certain flavoring agents advantageously incorporated into the effervescing dental treatment compositions as in addition to providing a flavoring benefit, they may also exhibit a useful antimicrobial property. Coming into consideration are, in particular, mint, and cinnamon which when present provide both a dual flavoring and antimicrobial property to the effervescing dental treatment compositions of which they form a part.

Non-limiting examples of colorants include edible dyes such as Red Food Dye #3, Coloring Yellow food No. 5 and Blue food coloring No. 1, yellow ferric oxide, red ferric oxide, brown iron oxide, black iron oxide, copper chlorophyll, copper and sodium chlorophyll, riboflavin, powdered green tea and the like. When present, the optional colorant provides 0.01-10% wt., of the total of the effervescing dental treatment compositions.

Non-limiting examples of binders include starches including those described above, which may have a multi-functional benefit. Useful binder include hydroxypropylcellulose, hydroxypropylmethylcellulose, crystalline cellulose, pregelatinized starch, polyvinylpyrrolidone, gum arabic powder, gelatin, pullulan, low-substituted hydroxypropylcellulose and the like. When present, the optional binder provides 0.1-50% wt., of the total of the effervescing dental treatment compositions. Particularly preferred binders and particularly preferred amounts of binders are disclosed with reference to one or more of the examples.

Non-limiting examples of a lubricant include stearic acid, stearic acid metal salt, sodium stearyl fumarate, fatty acid and sucrose ester, talc, hydrogenated oil, macrogol and the like. Examples of metal salt of stearic acid include magnesium stearate, calcium stearate, aluminum stearate and the like. When present, the optional lubricant provides 0.01-50% wt. of the total of the effervescing dental treatment compositions. Particularly preferred lubricants and particularly preferred amounts of lubricants are disclosed with reference to one or more of the examples.

Non-limiting examples of disintegrants include cross-linked polyvinylpyrrolidone (referred to as Polyplasdone or Crospovidone), a cross-linked sodium carboxymethyl cellulose (referred to as Croscarmellose sodium), sodium starch glycolate, low-substituted hydroxypropylcellulose, gelatinized starch, gelatin and the like. When present, the optional disintegrant provides 0.01-15% wt. of the total of the effervescing dental treatment compositions. Particularly preferred disintegrants and particularly preferred amounts of disintegrants are disclosed with reference to one or more of the examples.

The tableted effervescing dental treatment compositions in accordance with preferred embodiments of the present invention exhibit rapid disintegration in the oral cavity of person without the need for water or other drinks, in one embodiment, within 60 seconds, i.e., the tablet disintegrates in the saliva in the oral cavity for the ease of swallowing along with the saliva. In another embodiment, the tablet produced in accordance with the invention disintegrates in the oral cavity of a mammal within 10 seconds, within 20 seconds, within 30 seconds, within 40 seconds, within 50 seconds or within 60 seconds. Disintegration occurs without the need for water or other drinks. As the table disintegrates, the gas generator system is activated and effervesces thus ensuring good delivery of the remaining constituents within and preferably throughout the oral cavity.

As noted previously the compositions of the effervescing dental treatment compositions may provide in a variety of dosage forms, including one more of pearls, pastilles, noodles, semi agglomerated or agglomerated powders, as well as free-flowing particulates in a non-agglomerated or semi agglomerated form. Preferred are tablets as dosage forms which are readily dispensable, and insertable into the oral cavity. Such dosage forms are various but each dosage form should provide between about 50-250 grams, more preferably between about 75-225 grams, still more preferably between about 100-200 grams, yet more preferably between about 140-170 grams, in particular preferably about 145-155 grams of the effervescing dental treatment composition.

A further aspect of the present invention is directed toward a method of producing a tableted effervescing dental treatment composition, useful in providing a treatment benefit within the oral cavity, and in particular to dental surfaces within the mouth. The effervescing dental treatment compositions may be produced by conventional methods, and preferably include a tableting operation. Where a tableting operation is not practiced, and the effervescing dental treatment compositions take other non-tablet forms, the said composition is preferably provided in a packaged container which allows for protection of exposure to moisture prior to its use. In any embodiment, single packaging, such as sachets or flow wrapped individual dosage forms may be provided as a product, or in the alternative a larger container containing a greater quantity or plurality of tablets may also be provided.

In another aspect is provided a method of providing a treatment benefit to oral surfaces, in particular the reduction of plaque upon dental surfaces within the mouth, the method comprising the step of providing a tableted effervescing dental treatment composition as hereinafter described, and maintaining it within the oral cavity for sufficient time in order to provide reduction in dental plaque and/or to provide a freshening benefit to the oral cavity. Preferred embodiments of the effervescing dental treatment composition, preferably wherein the dosage form is three-dimensional body, exhibit rapid disintegration in the oral cavity of person without the need for water or other drinks. The retention of the effervesced dental treatment composition within the oral cavity provides for the distribution throughout the oral cavity, and in particular in and around all exposed surfaces of teeth, which are expected to comprise plaque upon their services. The interaction of the effervesce dental treatment composition functions to remediate the level of the plaque, and typically upon longer retention times within the oral cavity such as in excess of one minute, preferably in excess of two minutes, and increased to reduction in the level of the plaque is observed. In certain embodiments, it has also been surprisingly found that a whitening effect may also be provided by this method.

The method of the present invention may be practiced by any person, especially human child or adult. The method of the present invention may be practiced in the absence of typical lavatory facility (i.e., bathroom) wherein the mouth can be easily rinsed with water in a sink and the residue from normal toothbrushing and/or mouthwashing readily disposed after being expelled from the oral cavity. The method of the present invention is advantageously practiced in conjunction with physically and or psychologically handicapped persons who are unable to follow a normal regimen of oral hygiene. Such is particularly relevant to individuals who may be institutionalized, hospitalized, who have reduced motor functions, and who are not amenable to normal oral hygiene practices. In such an instance, one or more times a day, a dosage form of the effervescing dental treatment composition, advantageously in a tableted form, is introduced into the oral cavity. Following its effervescence, disintegration, the action of the composition upon the plaque provides an appreciable degree of plaque reduction while simultaneously, a breath freshening effect may be realized particularly wherein the composition includes useful amounts of flavorants. Such not only improves the oral health of the person being treated in such a manner, but also enhances the appearance of said person to others who have contact with them.

The foregoing features of the present invention, as well as further aspects of the invention are advantageously illustrated in one or more the following non-limiting examples.

### EXAMPLES

Various example compositions of effervescing dental treatment compositions were produced utilizing the following constituents, identified in the following Table A:

TABLE A
Constituent

| | |
|---|---|
| (a) aloe vera (Aloe | drained from gel capsules (250 mg/capsules) |
| barbadensis) | |
| (b) citric acid | citric acid (containing less than 2% silicon |
| | dioxide), anhydrous (food grade or USP grade) |
| (c) sodium bicarbonate | NaHCO₃, supplied as anhydrous food grade |
| | “baking soda” |
| (d) coconut oil/olive oil | Coconut oil/olive oil, blend at respective |
| blend | volume |
| (e) shitake mushroom | dried organic shitake mushroom |
| (f) cranberry extract | cranberry extract, which were drained from |
| | capsules (vaccinium macrocarpon) 50:1 |
| | concentrate equivalent to 15,000 mg of fresh |
| | cranberries per capsule and 100 mg Vitamin C |
| (g) black tea | black tea leaves, anhydrous (ex. Kenya) |
| (h) cornstarch | supplied as anhydrous cornstarch, food grade |
| (i) mint | fresh mint leaves, macerated and/or chopped |
| (j) cinnamon | cinnamon sticks, ground |
| (k) mint extract | liquid mint, commercial grade |
| (l) peppermint extract | liquid peppermint, commercial grade |
| (m) orange flavoring | liquid orange flavoring, commercial grade |
| (n) orange food coloring | food grade |
| (o) blue food coloring | food grade |
| (p) sweetener | Stevia ® sweetener, powder, food grace |
| (q) coconut oil | coconut oil, food grade |

All of the foregoing were food grade, and safely ingestible constituents. Unless indicated otherwise, all the foregoing, the constituents were all sourced from suppliers as “organic” graded constituents. Where a constituent is indicated to be “anhydrous”, such as to indicate that no added water was present, but naturally occurring water of crystallization (if present) are included and considered to be “anhydrous”.

### Example 1—Tablets

Tablets were produced first by forming a pre-tabletting mass from the following listed constituents identified on the foregoing Table 1; the resultant tablets formed from said pre-tabletting mass comprised the wt. % of the named constituents indicated on Table 1:

TABLE 1

| | pre-tabletting | | % wt. of Constituent |
|---|---|---|---|
| Constituent | mass (mg) | | in tablet composition |
| (a) aloe vera (Aloe barbadensis) | 20,000 | mg | 8.37 |
| (b) citric acid | 22,800 | mg | 9.54 |
| (c) sodium bicarbonate | 68,000 | mg | 28.46 |
| (d) coconut oil/olive oil blend | 40,800 | mg | 17.07 |
| (e) shitake mushroom | 31,200 | mg | 13.06 |
| (f) cranberry extract | 20,000 | mg | 8.37 |
| (g) black tea | 4,000 | mg | 1.67 |
| (h) cornstarch | 20,000 | mg | 8.37 |
| (i) mint | 8,000 | mg | 3.34 |
| (j) cinnamon | 4,140 | mg | 1.73 |

These tablets were produced in accordance with the following method steps.

Into a first container (bowl) was introduced the (h) cornstarch, the (b) citric acid, part of the (c) sodium bicarbonate all of which were in a pulverant form; these substantially dry powdered materials were mixed and homogenously blended. To this was added, and mixed into foregoing the (d) coconut oil/olive oil blend, and remainder of the (c) sodium bicarbonate.

Into a second container (bowl) was introduced the (a) aloe vera gel to which was added the (f) cranberry extract, which provided an equivalent of 30,000 mg of fresh cranberries and 200 mg Vitamin C.

Separately, (e) Shitake mushrooms were ground into a fine powder using a manually operated grinder followed by an electrically operated mini grinder, yielding 2 tablespoons of the ground mushrooms. The (g) black tea was also ground yielding 1 teaspoon of a fine powder which was then combined with the 2 tablespoons of the ground mushrooms.

The ground black tea, organic Shitake mushrooms were combined with the contents of the second container and thoroughly mixed to ensure a homogenous distribution. Thereafter these mixed constituents were combined with the contents of the first container first, by hand mixing, and then blended together until a smooth paste was obtained. From this paste, tablets (pastilles) were produced, first by using a ⅛ teaspoon measuring spoon to provide aliquots of the paste, which were thereafter first manually pressed using a mixing spatula at the open and of the measuring spoon and thereafter, these are delivered to the surface of wax paper to dry, for 48 hours in an ambient temperature environment (approximately 20° C.) which were subsequently frozen in residential refrigerator for 2-3 hours.

When a tablet thus formed was placed into the oral cavity of a human subject, who had not ingested any liquids immediately prior to placement into the oral cavity, it quickly disintegrated and effervesced, and this caused distribution of the tablet's contents within the oral cavity, and onto tooth surfaces and gingival tissue.

### Example 2—Tablets

Compositions used to form the tablets according to Example 1 were produced, and formed into tablets in the same manner. After allowing these tablets to thaw, the tablets were easily pulverized into a fine powder. Tubes were formed using food grade aluminum foil having an inner diameter of approximately 14 inch and lengths of approximately 4 inches. One end of each tube was crimped. Sufficient fine powder was introduced into each of several tubes, which are thereafter sealed. A first such filled tube was baked at 200° F. for 15 minutes. After allowing the baked tubes to cool to room temperature (approx. 20° C., 68° F.) and unrolled, the contents could be cut using a conventional kitchen knife into discs. It was observed that the resultant cut portions were somewhat soft. A second such filled tube was also baked, but at 250° F. for 20 minutes. Similarly, after cooled to room temperature, the contents could be cut using a conventional kitchen knife into discs. However, as observed that these were firmer than those baked at 200° F. for the shorter time interval. A third such filled tube was baked at 500° F., for two minutes. It was quickly observed that the contents were essentially turned into a charcoal appearing mass, and were not used and were discarded.

### Example 3: Testing

Tablets formed according to Example 1 were evaluated for their effect in human subjects. Eight (8) volunteer subjects were first treated by applying Reveal Flavored Disclosing Solution Concentrate (ex. Henry Schein Corp.) according to label directions. Each subject had a quantity of this material applied by a registered dental hygienist in a clinically appropriate manner to surfaces of their teeth and thereafter the amount of plaque revealed using this product was noted for each subject and classified by the dental hygienist using the scaled rankings of “mild”, “moderate” or “severe”. Such was undertaken for each subject, after a midday meal and following only a gross debridement done with a toothbrush. For each subject, one tablet of the composition of the invention was placed on the tongue and allowed to dissolve in the oral cavity. Immediately upon touching saliva, the tablet began to foam. In all eight of the subjects it took approximately one minute for the tablet to completely dissolve but subjects noted that the tablet appeared to continue foaming for an approximate two minute duration after which each subject spit out any residue. Thereafter, the same registered dental hygienist evaluated the thus treated subject, and by comparison with pretreatment results rated the efficacy of the treatment. Considering all eight subjects, an approximate 20% decrease in plaque on tooth surfaces was observed. Thereafter, the general health of each subject was monitored. No subject reported any adverse effects, i.e., either sensorially within the mouth, or any gastrointestinal distress.

### Example 4—Testing (II)

Tablets formed according to Example 2, which had been baked at 250° F. for 20 minutes before being cut were used in the testing protocol described in foregoing Example 3, with the added difference in that subjects were instructed not to brush their teeth for two days prior to the evaluation. As before, upon placement on the tongue the tablet immediately began to foam and took approximately one minute to dissolve. Each of the eight subjects also reported that a tingling effect persisted to approximately a full three minutes following the introduction of the tablet. After spitting out any residue, the oral cavity was rinsed with tap water which was also ejected. Afterwards, the same registered dental hygienist who had pretreated teeth, now evaluated the post treated teeth of each of the subjects to evaluate the reduction in observable plaque. The average of all of these eight subjects was a perceived reduction of approximately 35%. No subject reported any adverse effects, i.e., either sensorially within the mouth, or any gastrointestinal distress.

### Example 5—Testing (III)

The protocols according to example 4 was repeated with further subjects, each of whom had similarly first treated using the Reveal Flavored Disclosing Solution Concentrate applied by a registered dental hygienist who immediately thereafter evaluated the percentage of observable plaque as disclosed by the aforesaid product. According to the present example, after the introduction of the tablets formed according to example 2, is further subjects were asked to also agitate, viz., “swish”, the effervescing contents of the tablets to ensure enhanced distribution within the oral cavity, including the inter-tooth recesses for between 2-3 minutes. Afterwards, the residue if any, was spit out and after a brief aqueous rinse using tap water, the subject was reevaluated by the same registered dental hygienist who evaluated the effects of said treatment. In each instance, a significant reduction of plaque was reported.

Additionally, as noted that it was observed that in each of Examples 3, 4 and 5 that in addition to a measure reduction in plaque production additionally and observable bleaching or whitening effect also occurred.

### Example 6—Tablets

Further tablet compositions (“Tablets 6.0”) were formed, using the protocols of Examples 1 and 2. The compositions of these tablets were similar but added several additional constituents to those previously identified on Table 1, namely: (i) mint, as fresh whole leaves and (j) cinnamon.

The constituents and their amounts used to form these tablets was as follows:

TABLE 2

| Constituent | pre-tabletting mass (mg) | |
|---|---|---|
| (a) aloe vera (Aloe | 20,000 | mg |
| barbadensis) | | |
| (b) citric acid | 3 capsules, without exterior | |
| | shell + 2 tablespoons | |
| (c) sodium bicarbonate | 68,000 | mg |
| (d) coconut oil/olive oil blend | 6 | tablespoons |
| (e) shitake mushroom | 31,200 | mg |
| (f) cranberry extract | 20,000 | mg |
| (g) black tea | 4,000 | mg |
| (h) cornstarch | 20,000 | mg |
| (i) mint | 8,000 | mg |
| (j) cinnamon | 4,140 | mg |

As in Example 1, the constituents were (a)-(h) were prepared and described generally in the manner as previously disclosed however, according to the following protocol. Into a first container (bowl) was mixed (b) citric acid, 2 tablespoons, (h) cornstarch 2 tablespoons, and 1 cup sodium bicarbonate, which were well mixed to ensure homogeneity. To a second container (bowl) were mixed (a) aloe vera, the contents of 6 drained capsules, (b) citric acid, the contents of 3 capsules without exterior shells, (d) coconut oil/olive oil blend, 3 tablespoons, (e) shitake mushrooms, 2 tablespoons, pulverized, (f) cranberry extract, the content of 4 capsules, drained, (g) black tea, 1 tablespoon, pulverized and (d) coconut oil/olive oil blend, 3 tablespoons, which were well mixed to ensure homogeneity. The contents of the first bowl and the second bowl were mixed together, thereafter mixed in a blender and formed into a smooth paste, aliquots were then formed using various sized teaspoons, namely ⅛ teaspoon, 14 teaspoon, ⅕ teaspoon and 1 teaspoon measuring spoons were deposited onto a wax paper sheet, allowed to dry for 24 hours in a room temperature environment. The formed tablets (“tablets 6.0”) were allowed to rest in a quiescent state, for three weeks at room temperature. Thereafter, each of the tablets were examined for physical and visual attributes. There was no indication of any softness, the growth of mildew or mold, or any other degenerate of state visible upon visual inspection, without any magnification.

A second set of tablets (“Tablets 6.1”) was produced as identified immediately above, according to the same protocols. The compositions of the tablets differed only in that in addition to the measured amount of indicated constituents of Table 1, during production of the second set of tablets there is added to the second container the further constituents (i) mint, as fresh whole leaves and (j) cinnamon. As before the constituents were used to form a smooth paste as described previously.

A portion of the smooth paste comprising constituents (a)-(j), viz, the second set of tablets' composition, was baked at 250° F. for 20 minutes. It was observed that this did not solidify the tablets, but they did remain as an agglomerate. Baked tablets based on aliquots of ⅛ tablespoon and 14 tablespoon were separately introduced into the oral cavity. Farming of both of the tablets was observed to be good, but more pronounced foaming resulted with the larger, 14 tablespoon tablet.

The remaining second set of tablets (Tablets 6.1) was rebaked at 250° F. for 20 minutes, after having been retained in quiescent state, at room temperature, for 72 hours, to allow for further drying in this ambient environment. The rebaked tablets were firm to the touch. Upon admission to the oral cavity, they foamed effectively, and had an observable, slight cinnamon flavor.

Several further sets of tablets, formed from pre-tabletting masses as indicated on the following Table 3 were also formed, with the indicated constituents of the pre-tabletting masses as is reported here:

TABLE 3

| | Batch #3 | | Batch #4 | | Batch #5 | |
|---|---|---|---|---|---|---|
| Constituent | Tablets 6.2 | | Tablets 6.3 | | Tablets 6.4 | |
| (a) aloe vera (Aloe | 20,000 | mg | 20,000 | mg | 20,000 | mg |
| barbadensis) | | | | | | |
| (b) citric acid | 3 capsules, | | 22,800 | mg | 22,800 | mg |
| | without | | | | | |
| | exterior | | | | | |
| | shell + 2 | | | | | |
| | tablespoons | | | | | |
| (c) sodium | 68,000 | mg | 68,000 | mg | 68,000 | mg |
| bicarbonate | | | | | | |
| (d) coconut oil/olive | 81,600 | mg | 40,800 | mg | — | |
| oil blend | | | | | | |
| (e) shitake | 31,200 | mg | 31,200 | mg | 31,200 | mg |
| mushroom | | | | | | |
| (f) cranberry extract | 4 capsules, | | 2 capsules, | | 2 capsules, | |
| | drained | | drained | | drained | |
| (g) black tea | 4,000 | mg | 4,000 | mg | 4,000 | mg |
| (h) cornstarch | 20,000 | mg | 15,000 | mg | 15,000 | mg |
| (i) mint | 1 tablespoon, | | ½ tablespoon, | | | |
| | chopped | | chopped | | | |
| (j) cinnamon | 4,140 | mg | 2,070 | mg | 2,070 | mg |
| (k) mint extract | ¼ | teaspoon | ½ | teaspoon | | |
| (l) peppermint | — | | — | | ¼ | teaspoon |
| extract | | | | | | |
| | Batch #6 | | Batch #7 | | Batch #8 | |
| Constituent | Tablets 6.5 | | Tablets 6.6 | | Tablets 6.7 | |
| (a) aloe vera (Aloe | 10,000 | mg | 10,000 | mg | 6,660 | mg |
| barbadensis) | | | | | | |
| (b) citric acid | 5,700 | mg | 5,700 | mg | 7,600 | mg |
| (c) sodium | 25,500 | mg | 25,500 | mg | 25,500 | mg |
| bicarbonate | | | | | | |
| (d) coconut oil/olive | 40,800 | mg | — | | — | |
| oil blend | | | | | | |
| (e) shitake | 1 head, | | ½ head, | | ½ head, | |
| mushroom | pulverized | | pulverized | | pulverized | |
| (f) cranberry extract | 2 capsules, | | 2 capsules, | | 2 capsules, | |
| | drained | | drained | | drained | |
| (g) black tea | 2,000 | mg | 2,000 | mg | 2,000 | mg |
| (h) cornstarch | 7,500 | mg | 7,500 | mg | 5,000 | mg |
| (j) cinnamon | 690 | mg | — | | — | |
| (l) peppermint | ¼ | teaspoon | — | | — | |
| extract | | | | | | |
| (m) orange flavoring | — | | 1 | teaspoon | — | |
| (n) orange food | — | | 1 | teaspoon | — | |
| coloring | | | | | | |
| (o) blue food | ⅛ | teaspoon | 1 | teaspoon | — | |
| coloring | | | | | | |

| (p) sweetener | — | — | | — | |
|---|---|---|---|---|---|
| (q) coconut oil | — | ½ | tablespoon | ¼ | tablespoon |

The various tablets of Table 3 were formed using the protocols disclosed in Examples 1 and 2.

The smooth paste having the formulation of Tablets 6.2 was dehydrated in a 14 cup for 10 hours, which is open to the ambient environment and at room temperature. A further quantity of this same smooth paste was dehydrated in a 12 cup for 48 hours, which was open to the ambient environment and at room temperature. The paste was baked, as per the conditions and protocols outlined in Example 2, and thereafter formed into tablets.

The smooth paste of having the formulation of Tablets 6.3 This paste was baked, as per the conditions and protocols outlined in Example 2, and thereafter formed into tablets which were dehydrated at 135° F., later, baked at 250° F. for 20 minutes.
35 g—10 additional mint extract drops
35 g—10 additional organic extract drops

A first aliquot of the smooth paste having the formulation of Tablets 6.4 was dehydrated at 150° F., and thereafter The paste was baked, as per the conditions and protocols outlined in Example 2, and thereafter formed into tablets. A second aliquot of the smooth paste having the formulation of Tablets 6.4 was baked at 250° F. for 20 minutes; after baking it was allowed to air dry at room temperature.

The smooth paste having the formulation of Tablet 6.5 was baked, as per the conditions and protocols outlined in Example 2. The resultant powder obtained was compressed into ½ teaspoon tablets, using a hand-held tablet press.

The smooth paste having the formulation of Tablet 6.6 was baked, as per the conditions and protocols outlined in Example 2. The resultant material was moist, and was formed into ½ teaspoon tablets which remain their structural integrity. These tablets were thereafter baked at 200° F. for 20 minutes, to ensure thorough drying.

The smooth paste having the formulation of Tablet 6.7 was baked, as per the conditions and protocols outlined in Example 2. Thereafter the resultant composition was formed into ½ teaspoon tablets in a tablet press; the resultant tablets maintaining their structural integrity.

1. A tableted effervescing dental treatment composition comprising:
a gas generator system which provides an effervescent effect;
an edible oil or blend of edible oils;
a natural starch;
aloe vera (Aloe barbadensis);
Shitake mushroom;
cranberry extract;
tea leaves;
wherein the composition exhibits rapid disintegration in the oral cavity of person without the need for water or other drinks.

2. The tableted effervescing dental treatment composition of claim 1 in a dosage form, selected from: pearls, pastilles, noodles, semi agglomerated or agglomerated powders, and free-flowing particulates in a non-agglomerated or semi agglomerated form.

3. The tableted effervescing dental treatment composition of claim 2 wherein the dosage form is insertable into the oral cavity.

4. The tableted effervescing dental treatment composition of claim 2 wherein the dosage form has a mass of between about 50-250 grams.

5. A method of producing a tableted effervescing dental treatment composition according to claim 1, comprising the steps of:
forming a mixture which comprises the gas generator system which provides an effervescent effect; the edible oil or blend of edible oils; the natural starch; the aloe vera (Aloe barbadensis); the Shitake mushroom; the cranberry extract; and the tea leaves;
forming a dosage form from the mixture.

6. The method of claim 5, which comprises the further step of providing a dosage form of the tableted effervescing dental treatment composition in a package.

7. A method of providing a treatment benefit to oral surfaces, in particular the reduction of plaque upon dental surfaces within the mouth, the method comprising the step of providing a tableted effervescing dental treatment composition according to claim 1, and maintaining it within the oral cavity for sufficient time in order to provide reduction in dental plaque and/or to provide a freshening benefit to the oral cavity.
